(19) United States
(12) Patent Application Publication
(10) Pub. No.: US 2025/0129357 A1
(43) Pub. Date: Apr. 24, 2025

(54) VALENCENE SYNTHASE MUTANT AND VALENCENE HIGH-YIELD STRAIN

(71) Applicant: WUHAN HESHENG TECHNOLOGY CO., LTD., Wuhan (CN)

(72) Inventor: Ziling YE, Wuhan (CN)

(21) Appl. No.: 18/728,070

(22) PCT Filed: Dec. 28, 2022
(86) PCT No.: PCT/CN2022/142660

§ 371 Date: Jul. 10, 2024

(30) Foreign Application Priority Data
Jan. 11, 2022 (CN) 202210027320.0

(51) Int. Cl. C12N 9/88; C12N 9/04; C12N 9/10; C12N 9/12; C12N 9/90; C12N 15/81; C12P 5/00; C12P 7/26; C12R 1/865

(52) U.S. Cl. CPC C12N 9/88; C12N 9/0006; C12N 9/1029; C12N 9/1085; C12N 9/1205; C12N 9/1229; C12N 9/90; C12N 15/81; C12P 5/002; C12P 7/26; C12R 2001/865; C12Y 101/01088; C12Y 203/01009; C12Y 203/0301; C12Y 205/01001; C12Y 207/01036; C12Y 207/04002; C12Y 401/01033; C12Y 402/03073; C12Y 503/03002

(57) ABSTRACT

The present disclosure belongs to the field of synthetic biology and relates to a valencene synthase mutant and a valencene high-yield strain. An enzyme for synthesizing valencene is derived from Eryngium glaciale, and upon enzyme directed evolution of the enzyme, a valencene synthase mutant with improved enzyme performance is obtained, and the yield of a strain containing the mutant is 3.15 times the yield of a strain containing a wild-type synthase. The valencene synthase mutant of the present disclosure enhances the capability of synthesizing valencene by a strain, and a powerful foundation is laid for the industrial production thereof. A high-yield strain for synthesizing valencene is constructed by using the valencene synthetase mutant, and the yield of a fermentation tank reaches 12.4 g/L, which is the highest level reported to date.

### FIELD OF THE INVENTION

The present disclosure belongs to the field of synthetic biology and relates to a valencene synthase mutant and a valencene high-yield strain.

### BACKGROUND OF THE INVENTION

Valencene is a sesquiterpene compound with citrus flavor. It is one of the most valuable terpenoids used in commercial scale. It is widely used as a flavoring agent in food and beverage fields and has high economic value. At present, valencene is mainly obtained from plant extraction, but this method is not economically feasible due to its low content in plants and high cost of extraction.

Recently, microbial cell factories have been widely used in the production of chemical compounds. At present, expression of valencene synthase in microorganisms can realize heterologous synthesis of valencene in microorganisms, but the yield level achieved is still very low, wherein the main factor is that the activity of currently existing valencene synthase is not high, and complex metabolic engineering is often aquired to obtain relatively high yield of valencene.

Therefore, obtaining valencene synthase with high activity is one of the main factors to achieve high yield of valencene.

### SUMMARY OF THE INVENTION

The propose of the present disclosure is providing high-performance valencene synthase mutants and the use thereof in the production of valencene. The propose of the present disclosure is also to provide a valencene high-yield strain and a method for achieving increased valencene yield.

To achieve the above propose, the present disclosure provides the following technical solution: The present disclosure provides a valencene synthase mutant, the wild-type of which is valencene synthase derived from Eryngium glaciale with a sequence as shown in SEQ ID NO.1. The valencene synthase mutant has a substitution of amino acid residue in at least one position selected from positions 533, 336, 196, 176, 306 and 325 compared to the wild-type valencene synthase, wherein the positions are defined with reference to SEQ ID NO.1; the valencene synthase mutant has increased enzyme activity compared to the wild-type valencene synthase.

In some embodiments, the valencene synthase mutant comprises at least one of I533V, R336K, H196R, D176E, R306K, and K325E mutations.

In some embodiments, the valencene synthase mutant comprises I533V mutation, and optionally at least one of R336K, H196R, D176E, R306K, and K325E mutations.

In some embodiments, the valencene synthase mutant comprises at least one of I533V and R336K mutations, and optionally at least one of H196R, D176E, R306K, and K325E mutations.

The present disclosure also provides a valencene synthase mutant, the valencene synthase mutant comprises any one of the following mutation sites compared to the wild-type valencene synthase (SEQ ID NO.1)(1) I533V, R336K(2) I533V, R336K, H196R, D176E(3) I533V, R336K, R306K(4) I533V, R336K, K325E(5) I533V, R336K, H196R, D176E, R306K, K325E; wherein the sites are defined with reference to SEQ ID NO.1.

In some embodiments, the amino acid sequence of the valencene synthase mutant is least 90%, at least 91%, at least 92%, at least 93%, at least 94%, at least 95%, at least 96%, at least 97%, at least 98%, or at least 99% identical to the sequences shown in SEQ ID NO. 1.

In some embodiments, the amino acid sequence of the valencene synthase mutant is as shown in SEQ ID NOs. 92-96.

In some embodiments, compared to wild-type valencene synthase (SEQ ID NO. 1), the valencene synthase mutant as shown in SEQ ID NO. 92 has I533V and R336K mutations; the valencene synthase mutant as shown in SEQ ID NO. 93 has I533V, R336K, H196R and D176E mutations; the valencene synthase mutant as shown in SEQ ID NO. 94 has I533V, R336K and R306K mutations; the valencene synthase mutant as shown in SEQ ID NO. 95 has I533V, R336K and K325E mutations; the valencene synthase mutant as shown in SEQ ID NO. 96 has I533V, R336K, H196R, D176E, R306K and K325E mutations.

In some embodiments, the amino acid sequence of the valencene synthase mutant is least 90%, at least 91%, at least 92%, at least 93%, at least 94%, at least 95%, at least 96%, at least 97%, at least 98%, or at least 99% identical to the sequences shown in SEQ ID NOs. 92-96.

The present disclosure also provides a gene encoding the valencene synthase mutant described above, the nucleotide sequences of which, in some embodiments, can be optimized based on codon preference of the host.

The present disclosure also provides a recombinant plasmid comprising the gene, wherein the recombinant plasmid can express the valencene synthase mutant after being transferred into a host cell.

The present disclosure also provides recombinant cells comprising the above genes, the host of which includes bacteria (such as Escherichia coli), fungi (e.g. yeast (such as Saccharomyces cerevisiae), actinomycetes, etc.).

Use of the valencene synthase mutant, gene, recombinant plasmid or recombinant cell in the production of valencene and nootkatone.

The present disclosure also provides a valencene high-yield strain comprising the gene encoding the valencene synthase mutant described above.

The inventors have found that acetoacetyl coenzyme A thiolase, hydroxymethylglutaryl CoA (HMG-CoA) synthase, hydroxymethylglutaryl CoA (HMG-CoA) reductase, mevalonate kinase, mevalonate-5-phosphate kinase, mevalonate pyrophosphate decarboxylase, and isoprene pyrophosphate isomerase belong to enzymes in the mevalonate pathway (MVA pathway), and in the mevalonate pathway isoprene pyrophosphate (IPP) and dimethylallyl pyrophosphate (DMAPP) can be synthesized, both of which can be used as precursors for synthesizing farnesyl pyrophosphate (FPP) under the catalysis of farnesyl pyrophosphate synthase, whereras FPP is a substrate in the biosynthesis of valencene (the synthetic route of valencene is shown in FIG. 1). Therefore, when the recombinant bacteria/fungi can express at least one of the enzymes in the mevalonate pathway and the farnesyl pyrophosphate synthase, the synthesis of FPP would be facilitated, and then the biosynthesis of valencene would be facilitated.

In some embodiments, the valencene high-yield strain comprises gene ERG20 encoding farnesyl pyrophosphate synthase.

In some embodiments, the valencene high-yield strain comprises at least one of the genes in mevalonate (MVA) pathway, wherein the genes in mevalonate (MVA) pathway comprise: gene ERG10 encoding acetoacetyl coenzyme A thiolase, gene ERG13 encoding HMG-CoA synthase, gene tHMG1 encoding HMG-CoA reductase, gene ERG12 encoding mevalonate kinase, gene ERG8 encoding mevalonate-5-phosphate kinase, gene MVD1 encoding mevalonate pyrophosphate decarboxylase, and gene IDI1 encoding isoprene pyrophosphate isomerase.

In some embodiments, the valencene high-yield strain comprises gene ERG20 encoding farnesyl pyrophosphate synthase and a gene in mevalonate pathway (MVA pathway).

In some embodiments, the copy numbers of the genes in MVA pathway and the farnesene pyrophosphate synthase gene in the valencene high-yield strain are ERG10, ERG13, tHMG1, ERG12, ERG8, MVD1, IDI1, ERG20=2, 2, 3, 2, 2, 2, 2, 2.

In some embodiments, the copy number of the gene encoding the valencene synthase mutant is 2 or 3, preferably 2.

In some embodiments, the host of the high-yield valencene strain is Saccharomyces cerevisiae.

In some embodiments, the GAL80 gene in the valencene high-yield strain is knocked out when the host is Saccharomyces cerevisiae.

Advantages and advantageous effects of the present disclosure:
(1) The present disclosure obtains a mutant of valencene synthase having significantly improved performance, and the valencene yield of the strain comprising the mutant is 3.15 times higher than that of the strain comprising the wild-type synthase. The valencene synthase mutant disclosed has laid a strong foundation for the industrial production of valencene.
(2) In the present disclosure, a valencene high-yield strain is constructed using the valencene synthase mutant, and the fermentor yield of the constructed strain reaches 12.4 g/L, which is the highest level reported to date.

### BRIEF DESCRIPTION OF THE DRAWINGS

FIG. 1 is a diagram for the synthetic route of valencene.

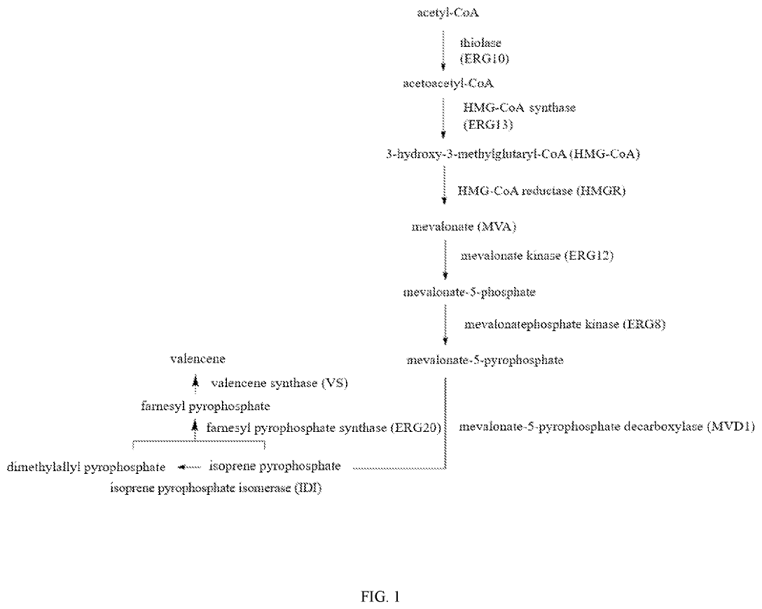

FIG. 2 shows the result for the amino acid alignment between EGVS and 5-epi-aristolochene synthase TEAS.

FIG. 3 is a graph of the result for the simulated EGVS and FPP docking.

FIG. 4 is a graph showing the result for the amino acid residue preference of the alignment between valencene synthase EgVS and its homologous sequence.

### DETAILED DESCRIPTION OF THE EMBODIMENTS

The following examples are used to further illustrate the present disclosure, but should not be construed as limiting the present disclosure. Any other changes, modifications, replacements, combinations, and simplifications made without departing from the spirit and principles of the present disclosure shall be equivalent substitutions, and are included in the protection scope of the present disclosure.

Unless otherwise specified, the technical means used in the examples are conventional means well known to those skilled in the art. The plasmids involved in the following examples are all well-known to those skilled in the art. If specific techniques or conditions are not indicated in the examples, the test is carried out according to the techniques or conditions described in the literature in the art or according to the product specification. Reagents or instruments used without indicating the manufacturer are commercially available conventional products.

### Example 1. Construction of Yeast Expression Vector

Plasmid pZY900 Related Features:

Δ LEU2: LEU2 (URA3)_TCYC1 LacZ_pGAL10pGAL1_ERG20_tERG20, promoter GAL1 and GAL10 control the expression of genes ERG20 and LacZ respectively, screening marker is Leu2, and the chromosome locus inserted is Leu2.

Specific construction process of plasmid pZY900: using the genome of Saccharomyces cerevisiae S288c as the template, amplify with primers 900-1F/1R, 900-2F/2R, 900-6F/6R and 900-7F/7R to obtain fragments 9001 (left homologous arm of Leu2), 9002 (terminator tTDH2), 9006 (gene ERG20 and terminator tERG20), 9007 (right homologous arm of Leu2) respectively; using the genome of Saccharomyces cerevisiae CEN.PK2-1D as the template, amplify with primers 900-3F/3R and 900-5F/5R to obtain fragments 9003 (terminator tCYC1) and 9005 (promoters pGAL1 and Pgal10) respectively; using pCAS as the template, amplify with primers 900-4F/4R to obtain fragments 9004 (nonsense gene, used for replacement of target gene); using pRS426 as the template, amplify with primers 900-8F/8R to obtain plasmid backbone (MssI restriction site, screening marker introduced). pZY900 was constructed by DNA assembly (Yeast assembly) in Saccharomyces cerevisiae, and then amplified in Escherichia coli. pZY900 was obtained after verified by enzyme digestion and sequencing. (For the construction of pCAS, see Zhang, Yueping et al. “A gRNA-tRNA array for CRISPR-Cas9 based rapid multiplexed genome editing in Saccharomyces cerevisiae.”) Nature communications vol. 10, 1 1053. 5 Mar. 2019, doi:10.1038/s41467-019-09005-3)₀

The sequences of the primers used to construct plasmid pZY900 are shown in Table 1 below:

TABLE 1

| Plasmid | Primer | Sequence (5′-3′) |
|---|---|---|
| pZY900 | 900-1F | actaaagggaacaaaagctggagctctagtagtttaaacataacgagaacacacagggg (SEQ ID NO. 4) |
| | 900-1R | cattaaagtaacttaaggagttaaatttaagcaaggattttcttaacttcttc (SEQ ID NO. 5) |
| | 900-2F | gaagttaagaaaatccttgcttaaatttaactccttaagttactttaatgatttag (SEQ ID NO. 6) |
| | 900-2R | tcgaaggctttaatttgcgcgaaaagccaattagtgtgata (SEQ ID NO. 7) |
| | 900-3F | tagtatcacactaattggcttttcgcgcaaattaaagccttcgagc (SEQ ID NO. 8) |
| | 900-3R | gggacgcgccctgtagcggctgaggtctcaacaggccccttttcctttg (SEQ ID NO. 9) |
| | 900-4F | catgatatcgacaaaggaaaaggggcctgttgagacctcagccgctacagggcgc (SEQ ID NO. 10) |
| | 900-4R | gaatttttgaaaattcaatataaatgtgagaccaccatgattacgccaagcg (SEQ ID NO. 11) |
| | 900-5F | taatcatggtggtctcacatttatattgaattttcaaaaattcttactttttttttg (SEQ ID NO. 12) |
| | 900-5R | atctctctctcctaatttctttttctgaagccattatagttttttctccttgacgttaaagt (SEQ ID NO. 13) |
| | 900-6F | ttaacgtcaaggagaaaaaactataatggcttcagaaaaagaaattagga (SEQ ID NO. 14) |
| | 900-6R | atgtacaaatatcataaaaaaagagaatctttttaaaaaaaatccttggactagtcacg (SEQ ID NO. 15) |
| | 900-7F | actagtccaaggattttttttaaaaagattctctttttttatgatatttgtacataaac (SEQ ID NO. 16) |
| | 900-7R | gcgccattcgccattcaggctgcgcaactgttgtttaaacgacaacgaccaagctcaca (SEQ ID NO. 17) |
| | 900-8F | gatgtgagcttggtcgttgtcgtttaaacaacagttgcgcagcctgaatg (SEQ ID NO. 18) |
| | 900-8R | cgatagcgcccctgtgtgttctcgttatgtttaaactactagagctccagcttttgttc (SEQ ID NO. 19) |

Plasmid pYH300 Related Features:

Δ LEU2: LEU2 (URA3)_TCYC1 LacZ_pGAL10pGAL1_ERG20 tERG20, promoter GAL1 and GAL10 control the expression of genes ERG20 and LacZ respectively, screening marker is Leu2, and the chromosome locus inserted is Leu2. Its difference with pZY900 is that the restriction site between homology arm and plasmid backbone is NotI.

Plasmid pYH300 was constructed by using pZY900 as the template, amplify with primer P48-F/R to obtain fragments between homologous arms, amplify with primer P49-F/R to obtain vector backbone comprising NotI restriction site, and obtain plasmid by methods for homologous recombination.

| | |
|---|---|
| P48-F | gaacaaaagctggagctctagtagcggccgcataacgagaacacacagggg (SEQ ID NO. 20) |
| P48-R | gcaactgttgcggccgcgacaacgaccaagctcacatcaa (SEQ ID NO. 21) |
| P49-F | gttgtcgcggccgcaacagttgcgcagcctga (SEQ ID NO. 22) |
| P49-R | tactagagctccagcttttgttc (SEQ ID NO. 23) |

### Example 2. Construction of Valencene Synthesis Vector

Among the present studies on valenene synthase, the most frequently studied is valencene synthase CnVS from Callitropsis nootkatensis mentioned in the literature (Beekwilder, Jules et al., “Valencia synthase from the heartwood of Nootka cypress (Callitropsis nootkatensis) for biotechnological production of valencia.” Plant biotechnology journal vol. 12, 2 (2014): 174-82. doi:10.1111/pbi.12124), and valencene synthase EgVS from Eryngium glaciale mentioned in patent US 2015/0007368 A1 has not been reported in literature. The wild-type valencene synthases from these two sources were compared in the first place. The coding sequences of CnVS and EgVS are synthesized after optimization according to Saccharomyces cerevisiae codons, and the nucleotide sequences are as shown in SEQ ID NO.2 and SEQ ID NO.3 respectively.

Gene-specific primer pair CnVS-F/R was designed, using synthesized gene (SEQ ID NO.2) as the template. CnVS gene fragment was obtained by PCR amplification using Prime STAR high fidelity enzyme of Takara Company. After gel recovery was carried out by using Tiangen gel recovery kit, it was ligated into BsaI cleaved yeast expression vector pYH300 by methods for homologous recombination using homologous recombination kit of Yeasen Company. After sequencing confirmation, the yeast expression vector comprising this gene was obtained and named pYH329.

Gene-specific primer pair EgVS-F/R was designed, using synthesized gene (SEQ ID NO.3) as the template. EgVS gene fragment was obtained by PCR amplification using Prime STAR high fidelity enzyme of Takara Company. After gel recovery was carried out by using Tiangen gel recovery kit, it was ligated into BsaI cleaved yeast expression vector pYH300 by methods for homologous recombination using homologous recombination kit of Yeasen Company. After sequencing confirmation, the yeast expression vector comprising this gene was obtained and named pYH327.

| Primer | 5′-3′ |
|---|---|
| CnVS-F | catgatatcgacaaaggaaaaggggcctgtttaaggaataataggttcaacaaagaact (SEQ ID NO. 24) |
| CnVS-R | tccaaaaaaaaagtaagaatttttgaaaattcaatataaatggctgaaatgttcaacgg (SEQ ID NO. 25) |
| EgVS-F | tacatgatatcgacaaaggaaaaggggcctgtttataatggaattggatcaaccaacaa (SEQ ID NO. 26) |
| EgVS-R | aaaagtaagaatttttgaaaattcaatataaatgtcattgaatgttttatctacatcag (SEQ ID NO. 27) |

### Example 3. Construction of a Valencene Synthesizing Strain

Plasmids pYH327 and pYH329 were linearized using NotI to obtain cleaved pYH327-NotI and pYH329-NotI fragments, which were transformed into competent yeast strain JCR27 by PEG/LiAC method respectively (construction of yeast strain JCR27 is described in Siemon, Thomas et al., “Semisynthesis of Plant-Derived Englerin A Enabled by Microbe Engineering of Guaia-6, 10(14)-diene as Building Block.” Journal of the American Chemical Society vol. 142, 6 (2020): 2760-2765. doi:10.1021/jacs.9bl2940). The positive strains obtained were named JGH29 and JGH31. Strain JGH29 is based on Saccharomyces cerevisiae CEN.PK2-1D with firstly enhanced MVA pathway followed by transferring a fragment comprising farnesyl pyrophosphate synthase gene and EgVS inside. Strain JGH31 is based on Saccharomyces cerevisiae CEN.PK2-1D with firstly enhanced MVA pathway followed by transferring a fragment comprising farnesyl pyrophosphate synthase gene and CnVS inside.

### Example 4. Shake Flask Fermentation of Strain JGH29 and JGH31

The strains JGH29 and JGH31 were seeded respectively into the seed culture medium (tryptone (20 g/L), yeast extract (10 g/L), glucose (20 g/L)) at 30° C., 200 rpm for 20-24 h, then transferred into the fermentation medium (tryptone (20 g/L), yeast extract (10 g/L), glucose (10 g/L), galactose (10 g/L)), followed by organic phase (n-dodecane or isopropyl myristate) covering 20% of the fermentation broth volume after transformation and fermentation at 30° C., 200 rpm for 72 h. GCMS detection showed that the yield of valencene of strain JGH29 was 78 mg/L and the yield of valencene of strain JGH31 was 22 mg/L. After this experiment, we found that valencene synthase from Eryngium glaciale has better performance.

### Example 5. Acquisition of Valencene Synthase Mutant Vector

Although above experiments proved that the valencene synthase from Eryngium glaciale has better performance, its yield is only less than 100 mg/L. The performance of valencene synthase needs to be further improved to make the yield of valencene reach the level for industrial production.

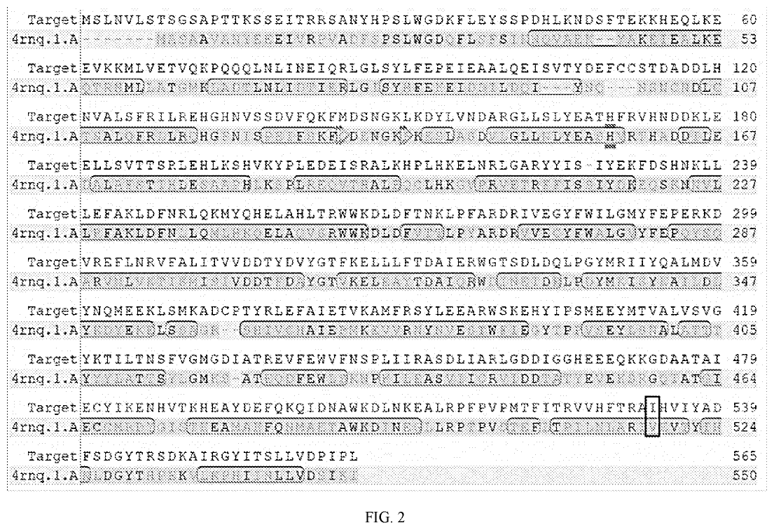

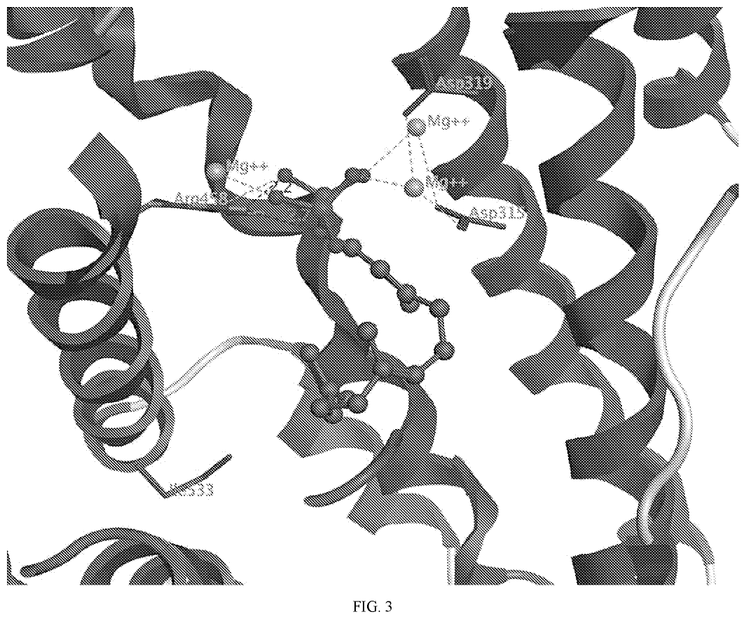

First, we used Swiss-model for three-dimensional structure simulation of the enzyme. The optimal template used was 4RNQ. The results for sequence alignment there between showed that the amino acid residue on the template 4RNQ corresponding to the 1533 site of the enzyme was V (FIG. 2). The structure simulation results showed that 1533 was located at the bottom of the substrate binding cavity (FIG. 3). Therefore, it was speculated that the mutation of I533V may increase the volume of the binding cavity, which was more suitable for substrate binding. Finally, the mutation was selected for experimental verification, and the valencene mutant I533V was constructed.

Using synthesized gene (SEQ ID NO.3) as the template, gene-specific primer pair P4-F/P50-R was designed. Partial gene fragment of EgVS was obtained by PCR amplification using Prime STAR high fidelity enzyme of Takara Company. The gene fragment of the rest of EgVS was obtained by PCR amplification with P51-F/P4-R. After gel recovery was carried out by using Tiangen gel recovery kit, they were ligated into BsaI cleaved yeast expression vector pYH300 by methods for homologous recombination using homologous recombination kit of Yeasen Company. After sequencing confirmation, the yeast expression vector comprising this gene was obtained and named pYH332.

| | |
|---|---|
| P4-F | tacatgatatcgacaaaggaaaaggggcctgtttataatggaattggatcaaccaacaa (SEQ ID NO. 28) |
| P50-R | tattacaagagttgttcatttcactagagctgttcatgttatctatgcagatttctcag (SEQ ID NO. 29) |
| P51-F | gtaaccatctgagaaatctgcatagataacatgaacagctctagtgaaatgaacaactc (SEQ ID NO. 30) |
| P4-R | aaaagtaagaatttttgaaaattcaatataaatgtcattgaatgttttatctacatcag (SEQ ID NO. 31) |

During the construction of pYH332, we unexpectedly obtained a plasmid comprising both I533V and R336K mutations, which was named pYH340. Both plasmids were integrated into yeast strain JCR27 after NotI linearization to obtain strains JGH37 and JGH44, the yields of valencene at the level of shake flask fermentation (conditions same as in Example 4) reached 69 mg/L and 100 mg/L, respectively, and the performance of the enzyme comprising the I533V and R336K mutations was improved compared to that of the wild-type EgVS.

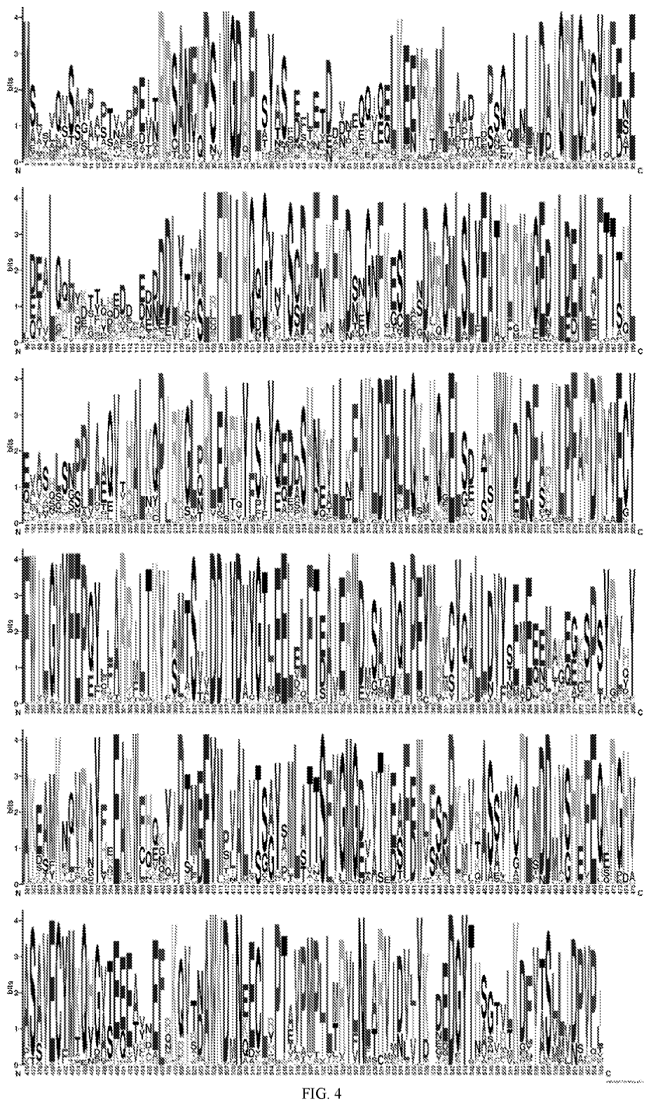

To further improve the performance of EgVS, amino acid residue preferences were demonstrated by homology alignment with EgVS (FIG. 4). Eight sites N81D, D176E, E216G, R306K, K325E, G347E, H491E, R350K were selected for site-directed mutagenesis of EgVS (I533V, R336K).

Using pYH340 plasmid (sequence comprising EgVS (I533V, R336K) mutation) as the template, gene-specific primer pair P4-F/P52-R was designed. Partial gene fragment of EgVS was obtained by PCR amplification using Prime STAR high fidelity enzyme of Takara Company. The gene fragment of the rest of EgVS was obtained by PCR amplification with P53-F/P4-R. After gel recovery was carried out by using Tiangen gel recovery kit, they were ligated into BsaI cleaved yeast expression vector pZY900 by methods for homologous recombination using homologous recombination kit of Yeasen Company. After sequencing confirmation, the yeast expression vector comprising the coding sequence of EgVS (I533V, R336K, N81D) was obtained and named pYH355.

Using pYH340 plasmid as the template, gene-specific primer pair P4-F/P54-R was designed. Partial gene fragment of EgVS was obtained by PCR amplification using Prime STAR high fidelity enzyme of Takara Company. The gene fragment of the rest of EgVS was obtained by PCR amplification with P55-F/P4-R. After gel recovery was carried out by using Tiangen gel recovery kit, they were ligated into BsaI cleaved yeast expression vector pZY900 by methods for homologous recombination using homologous recombination kit of Yeasen Company. After sequencing confirmation, the yeast expression vector comprising the coding sequence of EgVS (I533V, R336K, H196R, D176E) was obtained and named pYH356, in which the H196R mutation was accidentally introduced during the construction.

Using pYH340 plasmid as the template, gene-specific primer pair P4-F/P56-R was designed. Partial gene fragment of EgVS was obtained by PCR amplification using Prime STAR high fidelity enzyme of Takara Company. The gene fragment of the rest of EgVS was obtained by PCR amplification with P57-F/P4-R. After gel recovery was carried out by using Tiangen gel recovery kit, they were ligated into BsaI cleaved yeast expression vector pZY900 by methods for homologous recombination using homologous recombination kit of Yeasen Company. After sequencing confirmation, the yeast expression vector comprising the coding sequence of EgVS (I533V, R336K, E216G) was obtained and named pYH358.

Using pYH340 plasmid as the template, gene-specific primer pair P4-F/P58-R was designed. Partial gene fragment of EgVS was obtained by PCR amplification using Prime STAR high fidelity enzyme of Takara Company. The gene fragment of the rest of EgVS was obtained by PCR amplification with P59-F/P4-R. After gel recovery was carried out by using Tiangen gel recovery kit, they were ligated into BsaI cleaved yeast expression vector pZY900 by methods for homologous recombination using homologous recombination kit of Yeasen Company. After sequencing confirmation, the yeast expression vector comprising the coding sequence of EgVS (I533V, R336K, R306K) was obtained and named pYH361.

Using pYH340 plasmid as the template, gene-specific primer pair P4-F/P60-R was designed. Partial gene fragment of EgVS was obtained by PCR amplification using Prime STAR high fidelity enzyme of Takara Company. The gene fragment of the rest of EgVS was obtained by PCR amplification with P61-F/P4-R. After gel recovery was carried out by using Tiangen gel recovery kit, they were ligated into BsaI cleaved yeast expression vector pZY900 by methods for homologous recombination using homologous recombination kit of Yeasen Company. After sequencing confirmation, the yeast expression vector comprising the coding sequence of EgVS (I533V, R336K, K325E) was obtained and named pYH362.

Using pYH340 plasmid as the template, gene-specific primer pair P4-F/P62-R was designed. Partial gene fragment of EgVS was obtained by PCR amplification using Prime STAR high fidelity enzyme of Takara Company. The gene fragment of the rest of EgVS was obtained by PCR amplification with P63-F/P4-R. After gel recovery was carried out by using Tiangen gel recovery kit, they were ligated into BsaI cleaved yeast expression vector pZY900 by methods for homologous recombination using homologous recombination kit of Yeasen Company After sequencing confirmation, the yeast expression vector comprising the coding sequence of EgVS (533 R336K, G347E) was obtained and named pYH363.

Using pYH34O plasmid as the template, gene-specific primer pair P4-F/P64-R was designed. Partial gene fragment of EgVS was obtained by PCR amplification using Prime STAR high fidelity enzyme of Takara Company The gene fragment of the rest of EgVS was obtained by PCR amplification with P65-F/P4-R. After gel recovery was carried out by using Tiangen gel recovery kit, they were ligated into BsaJ cleaved yeast expression vector pZY900 by methods for homologous recombination using homologous recombination kit of Yeasen Company. After sequencing confirmation, the yeast expression vector comprising the coding sequence of EgVS (1533V, R336K, H491E) was obtained and named pYH372.

Using pYH34O plasmid as the template, gene-specific primer pair P4-F/P66-R was designed. Partial gene fragment of EgVS was obtained by PCR amplification using Prime STAR high fidelity enzyme of Takara Company The gene fragment of the rest of EgVS was obtained by PCR amplification with P67-F/P4-R. Aftergel recovery was carried out by using Tiangen gel recovery kit, they were ligated into BsaJ cleaved yeast expression vector pZY900 by methods for homologous recombination using homologous recombination kit of Yeasen Company. After sequencing confirmation, the yeast expression vector comprising the coding sequence of EgVS (1533V, R336K, R350K) was obtained and named pYH375.

| | |
|---|---|
| P52-R | aacaacaattgaatttgattgatgaaatccaaagattgggtttgt (SEQ ID NO. 32) |
| P53-F | acccaatctttggatttcatcaatcaaattcaattgttgttgtgg (SEQ ID NO. 33) |
| P54-R | cattttagagttcataacgaagataagttggaagaattgttgtcag(SEQ ID NO. 34) |
| P55-F | aacaattcttccaacttatcttcgttatgaactctaaaatgtgttgc (SEQ ID NO. 35) |
| P56-R | gaagcatccattgcataagggtttgaacagattgggtgcaaga (SEQ ID NO. 36) |
| P57-F | cttgcacccaatctgttcaaacccttatgcaatggatgcttca (SEQ ID NO. 37) |
| P58-R | tgttagagaattcttgaacaaagttttcgctttgatcactgtt (SEQ ID NO. 38) |
| P59-F | cagtgatcaaagcgaaaactttgttcaagaattctctaacatcctttc (SEQ ID NO. 39) |
| P60-R | acgatgtttacggtacttttgaagaattgttgttgtttactgatgc (SEQ ID NO. 40) |
| P61-F | agtaaacaacaacaattcttcaaaagtaccgtaaacatcgtatgtat (SEQ ID NO. 41) |
| P62-R | tgatttggatcaattgccagaatacatgagaatcatctatcaagctttg (SEQ ID NO. 42) |
| P63-F | tgatagatgattctcatgtattctggcaattgatccaaatcag (SEQ ID NO. 43) |
| P64-R | aggaaaaccatgttactaaggaagaagcatacgatgaattccaaaag (SEQ ID NO. 44) |
| P65-F | tggaattcatcgtatgcttcttccttagtaacatggttttccttgatgtaa (SEQ ID NO. 45) |
| P66-R | tcaattgccaggttacatgaaaatcatctatcaagctttgatggat (SEQ ID NO. 46) |
| P67-F | tcaaagcttgatagatgattttcatgtaacctggcaattgatc (SEQ ID NO. 47) |

### Example 6. Acquisition and Shake Flask Fermentation of Strains Comprising Valencene Synthase Mutants

Plasmids pYH355, 356, 358, 361, 362, 363, 372, 375 obtained above were linearized by MssJ and integrated into yeast strain JCR27 to obtain strains JGH55, 56, 57, 58, 59, 60, 63, 64.

The method of shake flask fermentation was same as in Example 4 above. Compared with strain JGH44, strains JGH57 and JGH60 comprising E216G and G347E mutations had significantly lower yields, which were l3 mg/L and 38 mg/L, respectively, strains JGH55, JGH63 and JGH64 comprising N81D, H491E and R350K mutations had slightly lower yields, which were 94 mg/L, 86 mg/L and 64 mg/L, respectively. However, strains JGH56, JGH58 and JGH59 comprising D176E (unexpected mutation H196R introduced during construction), R306K and K325E had increased yields, which were 117 mg/L, 143 mg/L and 114 mg/L, respectively. Plasmid pYH383 was constructed by combining the advantageous mutations. pYH383 was linearized by MssI and integrated into strain JCR27 to obtain strain JGH71, which had further improved yield. The yield of valencene reached 248 mg/L after 72 h shake flask fermentation of the strain comprising (I533V, R336K, H196R, D176E, R306K, K325E), which was 3.15 times higher than that of the wild-type. In addition, the proportion of by-products, valencene/aristolochene, decreased from 2.97:1 to 4.18:1. The yield of the final mutant was increased compared to that of the wild-type. Among these sites involving the increasing, except 1533 which was located inside the active pocket, all the other sites were located outside the active pocket and far away from the pocket, indicating that distal residues also had important effects on protein performance.

Construction of pYH383: using pYH362 as the template, gene-specific primer pair P4-F/P68-R was designed. Partial gene fragment of EgVS was obtained by PCR amplification using Prime STAR high fidelity enzyme of Takara Company. The gene fragment of the rest of EgVS was obtained by PCR amplification with P69-F/P4-R using pYH356 as the template. After gel recovery was carried out by using Tiangen gel recovery kit, they were ligated into BsaI cleaved yeast expression vector pZY900 by methods for homologous recombination using homologous recombination kit of Yeasen Company. After sequencing confirmation, the yeast expression vector comprising the coding sequence of EgVS (I533V, R336K, K325E, R306K, D176E, H196R) was obtained and named pYH383.

| | |
|---|---|
| P68-R | tgttagagaattcttgaacaaagttttcgctttgatcactgtt (SEQ ID NO. 48) |
| P69-F | cagtgatcaaagcgaaaactttgttcaagaattctctaacatcctttc (SEQ ID NO. 49) |

### Example 7. Metabolic Pathway Optimization Increases Valencene Production

In order to further increase the yield of valencene and to increase the copy number of valencene synthase, plasmids pYH384 and pYH385 were constructed.

Using that genome of Saccharomyces cerevisiae CEN.PK2-1D as the template, gene-specific primer pair P11-F/P11-R was designed, the left homologous arm of URA3 was obtained by PCR amplification; using commercial plasmid pRS423 as the template, amplify with primers P12-F/R to obtain histidine screening marker; using genome of CEN.PK2-1D as the template, amplify with primers PI3-F/R to obtain Tcyc1; using genome of Saccharomyces cerevisiae S288C as the template, amplify with primers P14-F/R to obtain Thmg1; using genome of CEN.PK2-1D as the template, amplify with primers P15-F/R to obtain pGAL1-pGAL10; using pYH383 as the template, amplify with primers P16-F/R to obtain the coding sequence of EgVS (I533V, R336K, K325E, R306K, D176E, H196R); using genome of CEN.PK2-1D as the template, amplify with primers P17-F/R to obtain tPGK1; using genome of CEN.PK2-1D as the template, amplify with primers P18-F/R to obtain right homologous arm of URA3; using commercial plasmid pRS426 as the template, amplify with primers P19-F/R to obtain vector backbone; pYH384 was constructed by DNA assembly (Yeast assembly) of above fragments in Saccharomyces cerevisiae, and then amplified in Escherichia coli. pYH384 was obtained after verified by enzyme digestion and sequencing. This plasmid comprises genes tHMG1 and EgVS (I533V, R336K, K325E, R306K, D176E, H196R).

Using that genome of Saccharomyces cerevisiae CEN.PK2-1D as the template, gene-specific primer pair P20-F/P20-R was designed, the left homologous arm of HIS3 was obtained by PCR amplification; using commercial plasmid pRS424 as the template, amplify with primers P21-F/R to obtain tryptophan screening marker; using genome of CEN.PK2-1D as the template, amplify with primers P22-F/R to obtain TAHD1; using pYH383 as the template, amplify with primers P23-F/R to obtain the coding sequence of EgVS (I533V, R336K, K325E, R306K, D176E, H196R); using genome of CEN.PK2-1D as the template, amplify with primers P24-F/R to obtain pGAL1-pGAL10; using genome of CEN.PK2-1D as the template, amplify with primers P25-F/R to obtain right homologous arm of tCPS1; using genome of CEN.PK2-1D as the template, amplify with primers P26-F/R to obtain right homologous arm of HIS3; using commercial plasmid pRS426 as the template, amplify with primers P27-F/R to obtain vector backbone; pYH385 was constructed by DNA assembly (Yeast assembly) of above fragments in Saccharomyces cerevisiae, and then amplified in Escherichia coli. pYH385 was obtained after verified by enzyme digestion and sequencing. The plasmid comprises gene EgVS (I533V, R336K, K325E, R306K, D176E, H196R).

| | |
|---|---|
| P11-F | attaaccctcactaaagggaacaaaagcgtttaaacacgcagataattccaggtatttt (SEQ ID NO. 50) |
| P11-R | aatacgactcactatagggcgaattgggtaccttcgtttcctgcaggtttttgt (SEQ ID NO. 51) |
| P12-F | caaaaacctgcaggaaacgaaggtacccaattcgccctatagtgag (SEQ ID NO. 52) |
| P12-R | gttttgggacgctcgaaggctttaatttgctcacagcttgtctgtaagcg (SEQ ID NO. 53) |
| P13-F | ttgtctgctcccggcatccgcttacagacaagctgtgagcaaattaaagccttcgagcg( SEQ ID NO. 54) |
| P13-R | gtttgaaagatgggtccgtcacctgcattaaatcctaaacaggccccttttcctttgtc (SEQ ID NO. 55) |
| P14-F | taattacatgatatcgacaaaggaaaaggggcctgtttaggatttaatgcaggtgacgg (SEQ ID NO. 56) |
| P14-R | gaatttttgaaaattcaatataaatggttttaaccaataaaacagtcat (SEQ ID NO. 57) |
| P15-F | gttttattggttaaaaccatttatattgaattttcaaaaattcttactttttttttgg (SEQ ID NO. 58) |
| P15-R | gtagataaaacattcaatgacattatagttttttctccttgacgttaaagt (SEQ ID NO. 59) |
| P16-F | cgtcaaggagaaaaaactataatgtcattgaatgttttatctacatcag (SEQ ID NO. 60) |
| P16-R | tgatctatcgatttcaattcaattcaatttataatggaattggatcaaccaaca (SEQ ID NO. 61) |
| P17-F | ctttgttggttgatccaattccattataaattgaattgaattgaaatcgatagatcaat (SEQ ID NO. 62) |
| P17-R | ttgaagctctaatttgtgagtttagtatacatgcatttacaacgaacgcagaattttcg (SEQ ID NO. 63) |
| P18-F | gtttaataactcgaaaattctgcgttcgttgtaaatgcatgtatactaaactcacaaat (SEQ ID NO. 64) |
| P18-R | gacggtcacagcttgtctgtgtttaaaccgtttaagggcaaatgtactct (SEQ ID NO. 65) |
| P19-F | agagtacatttgcccttaaacggtttaaacacagacaagctgtgaccgtc (SEQ ID NO. 66) |
| P19-R | tgcttcaaaatacctggaattatctgcgtgtttaaacgcttttgttccctttagtgagg (SEQ ID NO. 67) |
| P20-F | gcgcaattaaccctcactaaagggaacaaaagcgtttaaacaaacgtccagccacccat (SEQ ID NO. 68) |
| P20-R | atccgcttacagacaagctgtgatttagtatattcttcgaagaaatcacattactttat (SEQ ID NO. 69) |
| P21-F | ataaagtaatgtgatttcttcgaagaatatactaaatcacagcttgtctgtaagcggat (SEQ ID NO. 70) |
| P21-R | catgaggtcgctcttattgaccacacctctaccgggtacccaattcgccctatagtgag (SEQ ID NO. 71) |
| P22-F | cgcgtaatacgactcactatagggcgaattgggtacccggtagaggtgtggtcaataag (SEQ ID NO. 72) |
| P22-R | gttggttgatccaattccattataagcgaatttcttatgatttatgatttttattatta (SEQ ID NO. 73) |
| P23-F | aataaaaatcataaatcataagaaattcgcttataatggaattggatcaaccaacaaag (SEQ ID NO. 74) |
| P23-R | aaagtaagaatttttgaaaattcaatataaatgtcattgaatgttttatctacatcagg (SEQ ID NO. 75) |
| P24-F | atgtagataaaacattcaatgacatttatattgaattttcaaaaattcttacttttttt (SEQ ID NO. 76) |
| P24-R | tctttgactattcaatcattgcgctatagttttttctccttgacgttaaag (SEQ ID NO. 77) |
| P25-F | aacgtcaaggagaaaaaactatagcgcaatgattgaatagtcaaag (SEQ ID NO. 78) |
| P25-R | caactaactttttcccgtcctccatctcttatttgacacttgatttgacacttcttttt (SEQ ID NO. 79) |
| P26-F | taaaaaaaaaaagaagtgtcaaatcaagtgtcaaataagagatggaggacgggaaaaag (SEQ ID NO. 80) |
| P26-R | tgcagctcccggagacggtcacagcttgtctgtgtttaaacaaaccgtcagtggatgca (SEQ ID NO. 81) |
| P27-F | attggatctattgtctgcatccactgacggtttgtttaaacacagacaagctgtgaccg (SEQ ID NO. 82) |
| P27-R | aatcacaagaaatgggggctggacgtttgtttaaacgcttttgttccctttagtgagg (SEQ ID NO. 83) |

Plasmid pYH384 was linearized by MssI and integrated into strain JGH71 to obtain strain JGH72, which was based on CEN.PK2-1D and comprises a gene in mevalonate pathway, farnesene pyrophosphate synthase gene with copy numbers of ERG10, ERG13, tHMG1, ERG12, ERG8, MVD1, IDI1, ERG20=2, 2, 3, 2, 2, 2, 2, 2, and copy number of valencene synthase mutant encoding gene was 2.

Plasmid pYH385 was linearized with MssI and integrated into strain JGH72 to obtain strain JGH73, which was based on CEN.PK2-1D and comprises a gene in mevalonate pathway, farnesene pyrophosphate synthase gene with copy numbers of ERG10, ERG13, tHMG1, ERG12, ERG8, MVD1, IDI1, ERG20=2, 2, 3, 2, 2, 2, 2, 2, and copy number of valencene synthase mutant encoding gene was 3.

The strain was subjected to shake flask fermentation. The yield of strain JGH72 reached 393 mg/L when one copy of tHMG1 and valencene synthase was added, but the yield decreased when the quantity of valencene synthase was further increased, and the shake flask yield of strain JGH73 was 377 mg/L. This indicates that the strain comprising two copies of the valencene synthase mutant have higher yield. The strain JGH78 was obtained by further integrating pZY528 to knock out GAL80 to make up for the auxotrophic deficiency by eliminating galactose induction, and the yield reached 515 mg/L.

Construction of knockout cassette pZY528: Using CEN.PK2-1D genome as the template, fragment 5281 (comprising the left homologous arm of Gal80 site) was amplified by PCR with primers 5281-1F and 5281-1R, fragment 5284 (comprising right homologous arm of Gal80 site) was amplified by PCR with primers 5284-4F and 5284-4R; using plasmid pRS426-ura (ATCC87333) as the template, fragment 5282 (comprising uracil selection marker) was amplified by PCR with primers 5282-2F and 5282-2R; using plasmid pRS424 as the template, fragment 5283 (comprising tryptophan selection marker) was amplified by PCR with primers 5283-3F and 5283-3R; and fragment 5281, fragment 5282, fragment 5283 and fragment 5284 were ligated by OE-PCR using primers 5281-1F and 5284-4R to obtain pZY528.

| | |
|---|---|
| 5281-1F | cgcctgtctacaggataaagacgg (SEQ ID NO. 84) |
| 5281-1R | cgactcactatagggcgaattgggtacgacgggagtggaaagaacgg (SEQ ID NO. 85) |
| 5284-4F | taccgcacagatgcgtaaggagaaaataccgcatcaggaagcatcttgccctgtgcttg (SEQ ID NO. 86) |
| 5284-4R | aaatatgacccccaatatgagaaatt (SEQ ID NO. 87) |
| 5282-2F | tcccgttctttccactcccgtcgtacccaattcgccctatagtgag (SEQ ID NO. 88) |
| 5282-2R | atatatatagtaatgtcgtttcacagcttgtctgtaagcg (SEQ ID NO. 89) |
| 5283-3F | cgcttacagacaagctgtgaaacgacattactatatatataatataggaagcatttaat (SEQ ID NO. 90) |
| 5283-3R | gttcgctgcactgggggccaagcacagggcaagatgcttcctgatgcggtattttctcc (SEQ ID NO. 91) |

### Example 8. Fermentor Fermentation of Valencene High-Yield Strain

Fed-batch fermentation of the constructed strain JGH78 was carried out with reference to the ferment medium recited in the literature (van Hoek, P.; de Hulster, E.; van Di jken, J. P.; Pronk, J. T. Fermentative capacity in high-cell-density fed-batch cultures of baker's yeast Biotechnol. Bioeng 0.2000, 68, 517-523.), and a covering agent was added during the fermentation process to realize in situ extraction, wherein the covering agent was isopropyl myristate. The dissolved oxygen in fermentation process was controlled to be above 20%, pH to be 5, glucose concentration to be 1-2 g/L, and ethanol concentration to be less than 5 g/L. Finally, in a 15 L steel fermentor, the yield of valencene product of strain JGH78 reached 12.4 g/L, which is the highest level reported so far.

1. A valencene synthase mutant, wherein the valencene synthase mutant has a substitution of an amino acid residue in at least one position selected from positions 533, 336, 196, 176, 306 and 325 compared to a wild-type valencene synthase, wherein the amino acid sequence of the wild-type valencene synthase is as shown in SEQ ID NO.1; and the positions are defined with reference to SEQ ID NO.1; the valencene synthase mutant has increased enzyme activity compared to the wild-type valencene synthase;
preferably, the valencene synthase mutant comprises at least one of I533V, R336K, H196R, D176E, R306K, and K325E mutations;
preferably, the valencene synthase mutant comprises I533V mutation, and optionally at least one of R336K, H196R, D176E, R306K, and K325E mutations;
preferably, the valencene synthase mutant comprises at least one of I533V and R336K mutations, and optionally at least one of H196R, D176E, R306K, and K325E mutations;
preferably, the amino acid sequence of the valencene synthase mutant is at least 90%, at least 91%, at least 92%, at least 93%, at least 94%, at least 95%, at least 96%, at least 97%, at least 98%, or at least 99% identical to the sequence shown in SEQ ID NO: 1.

2. A valencene synthase mutant according to claim 1, wherein the valencene synthase mutant comprises any one of the following mutation sites compared to the wild-type valencene synthase:
I533V and R336K;
I533V, R336K, H196R and D176E;
I533V, R336K and R306K;
I533V, R336K and K325E;
I533V, R336K, H196R, D176E, R306K and K325E;
preferably, the amino acid sequence of the valencene synthase mutant is least 90%, at least 91%, at least 92%, at least 93%, at least 94%, at least 95%, at least 96%, at least 97%, at least 98%, or at least 99% identical to the sequences shown in SEQ ID NOs. 92-96; preferably, the amino acid sequence of the valencene synthase mutant is as shown in SEQ ID NOs. 92-96.

3. A gene encoding the valencene synthase mutant according to claim 1.

4. A recombinant plasmid comprising the gene according to claim 3.

5. A recombinant cell comprising the gene according to claim 3.

6. Use of the valencene synthase mutant according to claim 1, the gene encoding the valencene synthase mutant according to claim 1, and the recombinant plasmid or the recombinant cell comprising the gene encoding the valencene synthase mutant according to claim 1 in the production of valencene and nootkatone.

7. A valencene high-yield strain comprising the gene according to claim 3.

8. The valencene high-yield strain according to claim 7, wherein the valencene high-yield strain comprises gene ERG20 encoding farnesyl pyrophosphate synthase and/or at least one of the genes in mevalonate (MVA) pathway, wherein the genes in mevalonate (MVA) pathway comprise: gene ERG10 encoding acetoacetyl coenzyme A thiolase, gene ERG13 encoding HMG-CoA synthase, gene tHMG1 encoding HMG-CoA reductase, gene ERG12 encoding mevalonate kinase, gene ERG8 encoding mevalonate-5-phosphate kinase, gene MVD1 encoding mevalonate pyrophosphate decarboxylase, and gene IDI1 encoding isoprene pyrophosphate isomerase;
preferably, the valencene high-yield strain comprises gene ERG20 encoding farnesyl pyrophosphate synthase; and further comprises a gene in mevalonate pathway, wherein the gene in mevalonate pathway comprises: gene ERG10 encoding acetoacetyl coenzyme A thiolase, gene ERG13 encoding HMG-CoA synthase, gene tHMG1 encoding HMG-CoA reductase, gene ERG12 encoding mevalonate kinase, gene ERG8 encoding mevalonate-5-phosphate kinase, gene MVD1 encoding mevalonate pyrophosphate decarboxylase, and gene IDI1 encoding isoprene pyrophosphate isomerase.

9. The valencene high-yield strain comprising the gene according to claim 3 wherein the valencene high-yield strain comprises gene ERG20 encoding farnesyl pyrophosphate synthase; and further comprises a gene in mevalonate pathway, wherein the gene in mevalonate pathway comprises: gene ERG10 encoding acetoacetyl coenzyme A thiolase, gene ERG13 encoding HMG-CoA synthase, gene tHMG1 encoding HMG-CoA reductase, gene ERG12 encoding mevalonate kinase, gene ERG8 encoding mevalonate-5-phosphate kinase, gene MVD1 encoding mevalonate pyrophosphate decarboxylase, and gene IDI1 encoding isoprene pyrophosphate isomerase;
preferably, the copy numbers of the genes in MVA pathway and the farnesene pyrophosphate synthase gene are ERG10, ERG13, tHMG1, ERG12, ERG8, MVD1, IDI1, ERG20=2, 2, 3, 2, 2, 2, 2, 2; preferably, the copy number of the gene according to claim 3 is 2 or 3, preferably 2.

10. The valencene high-yield strain according to claim 7, wherein the host of the high-yield valencene strain is Saccharomyces cerevisiae.

11. The valencene high-yield strain according to claim 10, wherein the GAL80 gene in the valencene high-yield strain is knocked out.


SEQUENCE LISTING (96 sequences)

SEQ ID NO: 1 - protein, 565 aa, Eryngium glaciale
MSLNVLSTSGSAPTTKSSEITRRSANYHPSLWGDKFLEYSSPDHLKNDSFTEKKHEQLKEEVKKMLVETVQKPQQQLNLINEIQRLGLSYLFEPEIEAALQEISVTYDEFCCSTDADDLHNVALSFRILREHGHNVSSDVFQKFMDSNGKLKDYLVNDARGLLSLYEATHFRVHNDDKLEELLSVTTSRLEHLKSHVKYPLEDEISRALKHPLHKELNRLGARYYISIYEKFDSHNKLLLEFAKLDFNRLQKMYQHELAHLTRWWKDLDFTNKLPFARDRIVEGYFWILGMYFEPERKDVREFLNRVFALITVVDDTYDVYGTFKELLLFTDAIERWGTSDLDQLPGYMRIIYQALMDVYNQMEEKLSMKADCPTYRLEFAIETVKAMFRSYLEEARWSKEHYIPSMEEYMTVALVSVGYKTILTNSFVGMGDIATREVFEWVFNSPLIIRASDLIARLGDDIGGHEEEQKKGDAATAIECYIKENHVTKHEAYDEFQKQIDNAWKDLNKEALRPFPVPMTFITRVVHFTRAIHVIYADFSDGYTRSDKAIRGYITSLLVDPIPL

SEQ ID NO: 2 - DNA, 1770 bp, synthetic construct
  source 1..1770: mol_type = other DNA
atggctgaaatgttcaacggtaattcttctaatgatggttcatcatgtatgccagttaaggatgctttgaggagaactggcaatcatcaccctaacttgtggactgatgactttatacaatctttaaattctccatactcagattcttcataccataaacacagagaaattttaattgatgaaataagagatatgttttcaaacggtgagggtgatgagttcggtgttttggaaaatatatggtttgttgatgttgtccagaggttgggtattgatagacactttcaagaagagattaaaactgctttggattacatatacaaattctggaaccatgattcaattttcggtgatttgaatatggttgctttgggatttagaattttgagattaaatagatatgttgcttcttcagatgtctttaaaaaattcaaaggtgaggaaggtcaattctctggttttgaatcttctgaccaagatgcaaaattggaaatgatgttgaatttatataaggcttctgaattggattttccagatgaagatatattgaaagaagctagagcttttgcttctatgtacttgaaacatgtcattaaggaatacggtgatattcaagaatcaaagaacccattgttgatggagattgaatatacatttaagtacccttggaggtgtagattgccaagattggaggcttggaatttcattcatattatgagacaacaagattgtaacatatctttggcaaacaatttgtacaaaattcctaaaatatatatgaagaagattttggaattggctattttagatttcaatattttgcaatcacaacatcaacatgaaatgaagttgatttctacttggtggaaaaactcttctgctatacagttagatttctttcgtcatagacatattgaatcttacttctggtgggcttctcctttattcgaaccagagttttctacttgtagaattaattgtactaagttgtctactaagatgtttttgttggatgatatttacgatacttatggaactgttgaagaattgaagccatttacaacaactttaactagatgggatgtttctactgttgacaaccacccagattatatgaagattgctttcaatttttcttatgaaatttataaagaaattgcatcagaagcagagaggaagcacggtccatttgtctataaatatttgcaatcttgttggaaatcatacattgaggcttatatgcaagaagcagagtggatagcttcaaaccacattcccggttttgatgaatacttgatgaacggtgtcaaatcttctggtatgagaatattgatgattcatgctttgattttgatggatacaccattgtctgatgaaatattggaacaattagacataccatcttcaaagtcacaagccttattgtctttgattactaggttggttgatgatgttaaagatttcgaagatgaacaagctcatggtgaaatggcttcttctattgaatgttatatgaaggataaccacggttcaactagggaggacgcattgaattacttaaaaattaggattgaatcttgcgttcaagaattgaataaagaattattagagccttcaaacatgcacggttcttttcgtaatttatatttaaatgttggtatgagggttatcttttttatgttgaatgatggtgatttgtttacacattctaatagaaaagaaattcaagacgctattacaaagttctttgttgaacctattattccttaa

SEQ ID NO: 3 - DNA, 1698 bp, synthetic construct
  source 1..1698: mol_type = other DNA
atgtcattgaatgttttatctacatcaggttctgctccaactacaaagtcttcagaaatcactagaagatcagcaaactaccatccatctttatggggtgacaaatttttggaatactcttcaccagatcatttgaaaaatgattcttttacagaaaagaaacatgaacaattaaaagaagaagttaagaaaatgttggttgaaactgttcaaaagccacaacaacaattgaatttgattaatgaaatccaaagattgggtttgtcatacttattcgaaccagaaatcgaagctgcattgcaagaaatctcagttacttacgatgaattctgttgttctacagatgctgatgatttgcataacgttgcattgtcttttagaatcttgagagaacatggtcataatgtttcttcagatgtttttcaaaagtttatggattcaaacggtaaattgaaggattatttggttaatgatgctagaggtttgttatctttgtacgaagcaacacattttagagttcataacgatgataagttggaagaattgttgtcagttactacatctagattggaacatttgaagtcacatgttaagtacccattggaagatgaaatctctagagctttgaagcatccattgcataaggaattgaacagattgggtgcaagatactacatctcaatctatgaaaagtttgattctcataataagttgttattggaatttgctaaattagattttaatagattgcaaaagatgtatcaacatgaattggcacatttgacaagatggtggaaggatttggatttcactaataagttgccatttgctagagatagaatcgttgaaggttacttctggatcttgggcatgtacttcgaaccagaaagaaaggatgttagagaattcttgaacagagttttcgctttgatcactgttgttgatgatacatacgatgtttacggtacttttaaagaattgttgttgtttactgatgcaattgaaagatggggtacttctgatttggatcaattgccaggttacatgagaatcatctatcaagctttgatggatgtttacaaccaaatggaagaaaagttgtcaatgaaagcagattgtccaacatacagattggaattcgctatcgaaactgttaaggcaatgttcagatcttatttggaagaagctagatggtcaaaggaacattacatcccatctatggaagaatacatgacagttgcattagtttcagttggttacaagactatcttgacaaactctttcgttggtatgggtgacattgctactagagaagttttcgaatgggtttttaattcaccattaatcatcagagcttctgatttgattgcaagattaggtgacgatattggtggtcatgaagaagaacaaaagaaaggtgacgctgcaacagctatcgaatgttacatcaaggaaaaccatgttactaagcatgaagcatacgatgaattccaaaagcaaatcgataatgcttggaaagatttgaataaggaagcattgagaccatttccagttccaatgacttttattacaagagttgttcatttcactagagctatccatgttatctatgcagatttctcagatggttacacaagatctgataaggctatcagaggttacatcacttctttgttggttgatccaattccattataa

SEQ ID NO: 4 - DNA, 59 bp, synthetic construct
  source 1..59: mol_type = other DNA
actaaagggaacaaaagctggagctctagtagtttaaacataacgagaacacacagggg

SEQ ID NO: 5 - DNA, 53 bp, synthetic construct
  source 1..53: mol_type = other DNA
cattaaagtaacttaaggagttaaatttaagcaaggattttcttaacttcttc

SEQ ID NO: 6 - DNA, 56 bp, synthetic construct
  source 1..56: mol_type = other DNA
gaagttaagaaaatccttgcttaaatttaactccttaagttactttaatgatttag

SEQ ID NO: 7 - DNA, 41 bp, synthetic construct
  source 1..41: mol_type = other DNA
tcgaaggctttaatttgcgcgaaaagccaattagtgtgata

SEQ ID NO: 8 - DNA, 46 bp, synthetic construct
  source 1..46: mol_type = other DNA
tagtatcacactaattggcttttcgcgcaaattaaagccttcgagc

SEQ ID NO: 9 - DNA, 49 bp, synthetic construct
  source 1..49: mol_type = other DNA
gggacgcgccctgtagcggctgaggtctcaacaggccccttttcctttg

SEQ ID NO: 10 - DNA, 55 bp, synthetic construct
  source 1..55: mol_type = other DNA
catgatatcgacaaaggaaaaggggcctgttgagacctcagccgctacagggcgc

SEQ ID NO: 11 - DNA, 52 bp, synthetic construct
  source 1..52: mol_type = other DNA
gaatttttgaaaattcaatataaatgtgagaccaccatgattacgccaagcg

SEQ ID NO: 12 - DNA, 57 bp, synthetic construct
  source 1..57: mol_type = other DNA
taatcatggtggtctcacatttatattgaattttcaaaaattcttactttttttttg

SEQ ID NO: 13 - DNA, 62 bp, synthetic construct
  source 1..62: mol_type = other DNA
atctctctctcctaatttctttttctgaagccattatagttttttctccttgacgttaaagt

SEQ ID NO: 14 - DNA, 50 bp, synthetic construct
  source 1..50: mol_type = other DNA
ttaacgtcaaggagaaaaaactataatggcttcagaaaaagaaattagga

SEQ ID NO: 15 - DNA, 59 bp, synthetic construct
  source 1..59: mol_type = other DNA
atgtacaaatatcataaaaaaagagaatctttttaaaaaaaatccttggactagtcacg

SEQ ID NO: 16 - DNA, 59 bp, synthetic construct
  source 1..59: mol_type = other DNA
actagtccaaggattttttttaaaaagattctctttttttatgatatttgtacataaac

SEQ ID NO: 17 - DNA, 59 bp, synthetic construct
  source 1..59: mol_type = other DNA
gcgccattcgccattcaggctgcgcaactgttgtttaaacgacaacgaccaagctcaca

SEQ ID NO: 18 - DNA, 50 bp, synthetic construct
  source 1..50: mol_type = other DNA
gatgtgagcttggtcgttgtcgtttaaacaacagttgcgcagcctgaatg

SEQ ID NO: 19 - DNA, 59 bp, synthetic construct
  source 1..59: mol_type = other DNA
cgatagcgcccctgtgtgttctcgttatgtttaaactactagagctccagcttttgttc

SEQ ID NO: 20 - DNA, 51 bp, synthetic construct
  source 1..51: mol_type = other DNA
gaacaaaagctggagctctagtagcggccgcataacgagaacacacagggg

SEQ ID NO: 21 - DNA, 40 bp, synthetic construct
  source 1..40: mol_type = other DNA
gcaactgttgcggccgcgacaacgaccaagctcacatcaa

SEQ ID NO: 22 - DNA, 32 bp, synthetic construct
  source 1..32: mol_type = other DNA
gttgtcgcggccgcaacagttgcgcagcctga

SEQ ID NO: 23 - DNA, 23 bp, synthetic construct
  source 1..23: mol_type = other DNA
tactagagctccagcttttgttc

SEQ ID NO: 24 - DNA, 59 bp, synthetic construct
  source 1..59: mol_type = other DNA
catgatatcgacaaaggaaaaggggcctgtttaaggaataataggttcaacaaagaact

SEQ ID NO: 25 - DNA, 59 bp, synthetic construct
  source 1..59: mol_type = other DNA
tccaaaaaaaaagtaagaatttttgaaaattcaatataaatggctgaaatgttcaacgg

SEQ ID NO: 26 - DNA, 59 bp, synthetic construct
  source 1..59: mol_type = other DNA
tacatgatatcgacaaaggaaaaggggcctgtttataatggaattggatcaaccaacaa

SEQ ID NO: 27 - DNA, 59 bp, synthetic construct
  source 1..59: mol_type = other DNA
aaaagtaagaatttttgaaaattcaatataaatgtcattgaatgttttatctacatcag

SEQ ID NO: 28 - DNA, 59 bp, synthetic construct; identical to SEQ ID NO: 26
  source 1..59: mol_type = other DNA

SEQ ID NO: 29 - DNA, 59 bp, synthetic construct
  source 1..59: mol_type = other DNA
tattacaagagttgttcatttcactagagctgttcatgttatctatgcagatttctcag

SEQ ID NO: 30 - DNA, 59 bp, synthetic construct
  source 1..59: mol_type = other DNA
gtaaccatctgagaaatctgcatagataacatgaacagctctagtgaaatgaacaactc

SEQ ID NO: 31 - DNA, 59 bp, synthetic construct; identical to SEQ ID NO: 27
  source 1..59: mol_type = other DNA

SEQ ID NO: 32 - DNA, 45 bp, synthetic construct
  source 1..45: mol_type = other DNA
aacaacaattgaatttgattgatgaaatccaaagattgggtttgt

SEQ ID NO: 33 - DNA, 45 bp, synthetic construct
  source 1..45: mol_type = other DNA
acccaatctttggatttcatcaatcaaattcaattgttgttgtgg

SEQ ID NO: 34 - DNA, 46 bp, synthetic construct
  source 1..46: mol_type = other DNA
cattttagagttcataacgaagataagttggaagaattgttgtcag

SEQ ID NO: 35 - DNA, 47 bp, synthetic construct
  source 1..47: mol_type = other DNA
aacaattcttccaacttatcttcgttatgaactctaaaatgtgttgc

SEQ ID NO: 36 - DNA, 43 bp, synthetic construct
  source 1..43: mol_type = other DNA
gaagcatccattgcataagggtttgaacagattgggtgcaaga

SEQ ID NO: 37 - DNA, 43 bp, synthetic construct
  source 1..43: mol_type = other DNA
cttgcacccaatctgttcaaacccttatgcaatggatgcttca

SEQ ID NO: 38 - DNA, 43 bp, synthetic construct
  source 1..43: mol_type = other DNA
tgttagagaattcttgaacaaagttttcgctttgatcactgtt

SEQ ID NO: 39 - DNA, 48 bp, synthetic construct
  source 1..48: mol_type = other DNA
cagtgatcaaagcgaaaactttgttcaagaattctctaacatcctttc

SEQ ID NO: 40 - DNA, 46 bp, synthetic construct
  source 1..46: mol_type = other DNA
acgatgtttacggtacttttgaagaattgttgttgtttactgatgc

SEQ ID NO: 41 - DNA, 47 bp, synthetic construct
  source 1..47: mol_type = other DNA
agtaaacaacaacaattcttcaaaagtaccgtaaacatcgtatgtat

SEQ ID NO: 42 - DNA, 49 bp, synthetic construct
  source 1..49: mol_type = other DNA
tgatttggatcaattgccagaatacatgagaatcatctatcaagctttg

SEQ ID NO: 43 - DNA, 43 bp, synthetic construct
  source 1..43: mol_type = other DNA
tgatagatgattctcatgtattctggcaattgatccaaatcag

SEQ ID NO: 44 - DNA, 47 bp, synthetic construct
  source 1..47: mol_type = other DNA
aggaaaaccatgttactaaggaagaagcatacgatgaattccaaaag

SEQ ID NO: 45 - DNA, 51 bp, synthetic construct
  source 1..51: mol_type = other DNA
tggaattcatcgtatgcttcttccttagtaacatggttttccttgatgtaa

SEQ ID NO: 46 - DNA, 46 bp, synthetic construct
  source 1..46: mol_type = other DNA
tcaattgccaggttacatgaaaatcatctatcaagctttgatggat

SEQ ID NO: 47 - DNA, 43 bp, synthetic construct
  source 1..43: mol_type = other DNA
tcaaagcttgatagatgattttcatgtaacctggcaattgatc

SEQ ID NO: 48 - DNA, 43 bp, synthetic construct; identical to SEQ ID NO: 38
  source 1..43: mol_type = other DNA

SEQ ID NO: 49 - DNA, 48 bp, synthetic construct; identical to SEQ ID NO: 39
  source 1..48: mol_type = other DNA

SEQ ID NO: 50 - DNA, 59 bp, synthetic construct
  source 1..59: mol_type = other DNA
attaaccctcactaaagggaacaaaagcgtttaaacacgcagataattccaggtatttt

SEQ ID NO: 51 - DNA, 54 bp, synthetic construct
  source 1..54: mol_type = other DNA
aatacgactcactatagggcgaattgggtaccttcgtttcctgcaggtttttgt

SEQ ID NO: 52 - DNA, 46 bp, synthetic construct
  source 1..46: mol_type = other DNA
caaaaacctgcaggaaacgaaggtacccaattcgccctatagtgag

SEQ ID NO: 53 - DNA, 50 bp, synthetic construct
  source 1..50: mol_type = other DNA
gttttgggacgctcgaaggctttaatttgctcacagcttgtctgtaagcg

SEQ ID NO: 54 - DNA, 59 bp, synthetic construct
  source 1..59: mol_type = other DNA
ttgtctgctcccggcatccgcttacagacaagctgtgagcaaattaaagccttcgagcg

SEQ ID NO: 55 - DNA, 59 bp, synthetic construct
  source 1..59: mol_type = other DNA
gtttgaaagatgggtccgtcacctgcattaaatcctaaacaggccccttttcctttgtc

SEQ ID NO: 56 - DNA, 59 bp, synthetic construct
  source 1..59: mol_type = other DNA
taattacatgatatcgacaaaggaaaaggggcctgtttaggatttaatgcaggtgacgg

SEQ ID NO: 57 - DNA, 49 bp, synthetic construct
  source 1..49: mol_type = other DNA
gaatttttgaaaattcaatataaatggttttaaccaataaaacagtcat

SEQ ID NO: 58 - DNA, 58 bp, synthetic construct
  source 1..58: mol_type = other DNA
gttttattggttaaaaccatttatattgaattttcaaaaattcttactttttttttgg

SEQ ID NO: 59 - DNA, 51 bp, synthetic construct
  source 1..51: mol_type = other DNA
gtagataaaacattcaatgacattatagttttttctccttgacgttaaagt

SEQ ID NO: 60 - DNA, 49 bp, synthetic construct
  source 1..49: mol_type = other DNA
cgtcaaggagaaaaaactataatgtcattgaatgttttatctacatcag

SEQ ID NO: 61 - DNA, 54 bp, synthetic construct
  source 1..54: mol_type = other DNA
tgatctatcgatttcaattcaattcaatttataatggaattggatcaaccaaca

SEQ ID NO: 62 - DNA, 59 bp, synthetic construct
  source 1..59: mol_type = other DNA
ctttgttggttgatccaattccattataaattgaattgaattgaaatcgatagatcaat

SEQ ID NO: 63 - DNA, 59 bp, synthetic construct
  source 1..59: mol_type = other DNA
ttgaagctctaatttgtgagtttagtatacatgcatttacaacgaacgcagaattttcg

SEQ ID NO: 64 - DNA, 59 bp, synthetic construct
  source 1..59: mol_type = other DNA
gtttaataactcgaaaattctgcgttcgttgtaaatgcatgtatactaaactcacaaat

SEQ ID NO: 65 - DNA, 50 bp, synthetic construct
  source 1..50: mol_type = other DNA
gacggtcacagcttgtctgtgtttaaaccgtttaagggcaaatgtactct

SEQ ID NO: 66 - DNA, 50 bp, synthetic construct
  source 1..50: mol_type = other DNA
agagtacatttgcccttaaacggtttaaacacagacaagctgtgaccgtc

SEQ ID NO: 67 - DNA, 59 bp, synthetic construct
  source 1..59: mol_type = other DNA
tgcttcaaaatacctggaattatctgcgtgtttaaacgcttttgttccctttagtgagg

SEQ ID NO: 68 - DNA, 59 bp, synthetic construct
  source 1..59: mol_type = other DNA
gcgcaattaaccctcactaaagggaacaaaagcgtttaaacaaacgtccagccacccat

SEQ ID NO: 69 - DNA, 59 bp, synthetic construct
  source 1..59: mol_type = other DNA
atccgcttacagacaagctgtgatttagtatattcttcgaagaaatcacattactttat

SEQ ID NO: 70 - DNA, 59 bp, synthetic construct
  source 1..59: mol_type = other DNA
ataaagtaatgtgatttcttcgaagaatatactaaatcacagcttgtctgtaagcggat

SEQ ID NO: 71 - DNA, 59 bp, synthetic construct
  source 1..59: mol_type = other DNA
catgaggtcgctcttattgaccacacctctaccgggtacccaattcgccctatagtgag

SEQ ID NO: 72 - DNA, 59 bp, synthetic construct
  source 1..59: mol_type = other DNA
cgcgtaatacgactcactatagggcgaattgggtacccggtagaggtgtggtcaataag

SEQ ID NO: 73 - DNA, 59 bp, synthetic construct
  source 1..59: mol_type = other DNA
gttggttgatccaattccattataagcgaatttcttatgatttatgatttttattatta

SEQ ID NO: 74 - DNA, 59 bp, synthetic construct
  source 1..59: mol_type = other DNA
aataaaaatcataaatcataagaaattcgcttataatggaattggatcaaccaacaaag

SEQ ID NO: 75 - DNA, 59 bp, synthetic construct
  source 1..59: mol_type = other DNA
aaagtaagaatttttgaaaattcaatataaatgtcattgaatgttttatctacatcagg

SEQ ID NO: 76 - DNA, 59 bp, synthetic construct
  source 1..59: mol_type = other DNA
atgtagataaaacattcaatgacatttatattgaattttcaaaaattcttacttttttt

SEQ ID NO: 77 - DNA, 51 bp, synthetic construct
  source 1..51: mol_type = other DNA
tctttgactattcaatcattgcgctatagttttttctccttgacgttaaag

SEQ ID NO: 78 - DNA, 46 bp, synthetic construct
  source 1..46: mol_type = other DNA
aacgtcaaggagaaaaaactatagcgcaatgattgaatagtcaaag

SEQ ID NO: 79 - DNA, 59 bp, synthetic construct
  source 1..59: mol_type = other DNA
caactaactttttcccgtcctccatctcttatttgacacttgatttgacacttcttttt

SEQ ID NO: 80 - DNA, 59 bp, synthetic construct
  source 1..59: mol_type = other DNA
taaaaaaaaaaagaagtgtcaaatcaagtgtcaaataagagatggaggacgggaaaaag

SEQ ID NO: 81 - DNA, 59 bp, synthetic construct
  source 1..59: mol_type = other DNA
tgcagctcccggagacggtcacagcttgtctgtgtttaaacaaaccgtcagtggatgca

SEQ ID NO: 82 - DNA, 59 bp, synthetic construct
  source 1..59: mol_type = other DNA
attggatctattgtctgcatccactgacggtttgtttaaacacagacaagctgtgaccg

SEQ ID NO: 83 - DNA, 59 bp, synthetic construct
  source 1..59: mol_type = other DNA
aatcacaagaaatgggtggctggacgtttgtttaaacgcttttgttccctttagtgagg

SEQ ID NO: 84 - DNA, 24 bp, synthetic construct
  source 1..24: mol_type = other DNA
cgcctgtctacaggataaagacgg

SEQ ID NO: 85 - DNA, 47 bp, synthetic construct
  source 1..47: mol_type = other DNA
cgactcactatagggcgaattgggtacgacgggagtggaaagaacgg

SEQ ID NO: 86 - DNA, 59 bp, synthetic construct
  source 1..59: mol_type = other DNA
taccgcacagatgcgtaaggagaaaataccgcatcaggaagcatcttgccctgtgcttg

SEQ ID NO: 87 - DNA, 26 bp, synthetic construct
  source 1..26: mol_type = other DNA
aaatatgacccccaatatgagaaatt

SEQ ID NO: 88 - DNA, 46 bp, synthetic construct
  source 1..46: mol_type = other DNA
tcccgttctttccactcccgtcgtacccaattcgccctatagtgag

SEQ ID NO: 89 - DNA, 40 bp, synthetic construct
  source 1..40: mol_type = other DNA
atatatatagtaatgtcgtttcacagcttgtctgtaagcg

SEQ ID NO: 90 - DNA, 59 bp, synthetic construct
  source 1..59: mol_type = other DNA
cgcttacagacaagctgtgaaacgacattactatatatataatataggaagcatttaat

SEQ ID NO: 91 - DNA, 59 bp, synthetic construct
  source 1..59: mol_type = other DNA
gttcgctgcactgggggccaagcacagggcaagatgcttcctgatgcggtattttctcc

SEQ ID NO: 92 - protein, 565 aa, synthetic construct
MSLNVLSTSGSAPTTKSSEITRRSANYHPSLWGDKFLEYSSPDHLKNDSFTEKKHEQLKEEVKKMLVETVQKPQQQLNLINEIQRLGLSYLFEPEIEAALQEISVTYDEFCCSTDADDLHNVALSFRILREHGHNVSSDVFQKFMDSNGKLKDYLVNDARGLLSLYEATHFRVHNDDKLEELLSVTTSRLEHLKSHVKYPLEDEISRALKHPLHKELNRLGARYYISIYEKFDSHNKLLLEFAKLDFNRLQKMYQHELAHLTRWWKDLDFTNKLPFARDRIVEGYFWILGMYFEPERKDVREFLNRVFALITVVDDTYDVYGTFKELLLFTDAIEKWGTSDLDQLPGYMRIIYQALMDVYNQMEEKLSMKADCPTYRLEFAIETVKAMFRSYLEEARWSKEHYIPSMEEYMTVALVSVGYKTILTNSFVGMGDIATREVFEWVFNSPLIIRASDLIARLGDDIGGHEEEQKKGDAATAIECYIKENHVTKHEAYDEFQKQIDNAWKDLNKEALRPFPVPMTFITRVVHFTRAVHVIYADFSDGYTRSDKAIRGYITSLLVDPIPL

SEQ ID NO: 93 - protein, 565 aa, synthetic construct
MSLNVLSTSGSAPTTKSSEITRRSANYHPSLWGDKFLEYSSPDHLKNDSFTEKKHEQLKEEVKKMLVETVQKPQQQLNLINEIQRLGLSYLFEPEIEAALQEISVTYDEFCCSTDADDLHNVALSFRILREHGHNVSSDVFQKFMDSNGKLKDYLVNDARGLLSLYEATHFRVHNEDKLEELLSVTTSRLEHLKSRVKYPLEDEISRALKHPLHKELNRLGARYYISIYEKFDSHNKLLLEFAKLDFNRLQKMYQHELAHLTRWWKDLDFTNKLPFARDRIVEGYFWILGMYFEPERKDVREFLNRVFALITVVDDTYDVYGTFKELLLFTDAIEKWGTSDLDQLPGYMRIIYQALMDVYNQMEEKLSMKADCPTYRLEFAIETVKAMFRSYLEEARWSKEHYIPSMEEYMTVALVSVGYKTILTNSFVGMGDIATREVFEWVFNSPLIIRASDLIARLGDDIGGHEEEQKKGDAATAIECYIKENHVTKHEAYDEFQKQIDNAWKDLNKEALRPFPVPMTFITRVVHFTRAVHVIYADFSDGYTRSDKAIRGYITSLLVDPIPL

SEQ ID NO: 94 - protein, 565 aa, synthetic construct
MSLNVLSTSGSAPTTKSSEITRRSANYHPSLWGDKFLEYSSPDHLKNDSFTEKKHEQLKEEVKKMLVETVQKPQQQLNLINEIQRLGLSYLFEPEIEAALQEISVTYDEFCCSTDADDLHNVALSFRILREHGHNVSSDVFQKFMDSNGKLKDYLVNDARGLLSLYEATHFRVHNDDKLEELLSVTTSRLEHLKSHVKYPLEDEISRALKHPLHKELNRLGARYYISIYEKFDSHNKLLLEFAKLDFNRLQKMYQHELAHLTRWWKDLDFTNKLPFARDRIVEGYFWILGMYFEPERKDVREFLNKVFALITVVDDTYDVYGTFKELLLFTDAIEKWGTSDLDQLPGYMRIIYQALMDVYNQMEEKLSMKADCPTYRLEFAIETVKAMFRSYLEEARWSKEHYIPSMEEYMTVALVSVGYKTILTNSFVGMGDIATREVFEWVFNSPLIIRASDLIARLGDDIGGHEEEQKKGDAATAIECYIKENHVTKHEAYDEFQKQIDNAWKDLNKEALRPFPVPMTFITRVVHFTRAVHVIYADFSDGYTRSDKAIRGYITSLLVDPIPL

SEQ ID NO: 95 - protein, 565 aa, synthetic construct
MSLNVLSTSGSAPTTKSSEITRRSANYHPSLWGDKFLEYSSPDHLKNDSFTEKKHEQLKEEVKKMLVETVQKPQQQLNLINEIQRLGLSYLFEPEIEAALQEISVTYDEFCCSTDADDLHNVALSFRILREHGHNVSSDVFQKFMDSNGKLKDYLVNDARGLLSLYEATHFRVHNDDKLEELLSVTTSRLEHLKSHVKYPLEDEISRALKHPLHKELNRLGARYYISIYEKFDSHNKLLLEFAKLDFNRLQKMYQHELAHLTRWWKDLDFTNKLPFARDRIVEGYFWILGMYFEPERKDVREFLNRVFALITVVDDTYDVYGTFEELLLFTDAIEKWGTSDLDQLPGYMRIIYQALMDVYNQMEEKLSMKADCPTYRLEFAIETVKAMFRSYLEEARWSKEHYIPSMEEYMTVALVSVGYKTILTNSFVGMGDIATREVFEWVFNSPLIIRASDLIARLGDDIGGHEEEQKKGDAATAIECYIKENHVTKHEAYDEFQKQIDNAWKDLNKEALRPFPVPMTFITRVVHFTRAVHVIYADFSDGYTRSDKAIRGYITSLLVDPIPL

SEQ ID NO: 96 - protein, 565 aa, synthetic construct
MSLNVLSTSGSAPTTKSSEITRRSANYHPSLWGDKFLEYSSPDHLKNDSFTEKKHEQLKEEVKKMLVETVQKPQQQLNLINEIQRLGLSYLFEPEIEAALQEISVTYDEFCCSTDADDLHNVALSFRILREHGHNVSSDVFQKFMDSNGKLKDYLVNDARGLLSLYEATHFRVHNEDKLEELLSVTTSRLEHLKSRVKYPLEDEISRALKHPLHKELNRLGARYYISIYEKFDSHNKLLLEFAKLDFNRLQKMYQHELAHLTRWWKDLDFTNKLPFARDRIVEGYFWILGMYFEPERKDVREFLNKVFALITVVDDTYDVYGTFEELLLFTDAIEKWGTSDLDQLPGYMRIIYQALMDVYNQMEEKLSMKADCPTYRLEFAIETVKAMFRSYLEEARWSKEHYIPSMEEYMTVALVSVGYKTILTNSFVGMGDIATREVFEWVFNSPLIIRASDLIARLGDDIGGHEEEQKKGDAATAIECYIKENHVTKHEAYDEFQKQIDNAWKDLNKEALRPFPVPMTFITRVVHFTRAVHVIYADFSDGYTRSDKAIRGYITSLLVDPIPL